(19) United States
(12) Patent Application Publication
(10) Pub. No.: US 2002/0097884 A1
(43) Pub. Date: Jul. 25, 2002

(54) Variable noise reduction algorithm based on vehicle conditions

(76) Inventor: Douglas A. Cairns, Durham, NC (US)

Correspondence Address: COATS & BENNETT, PLLC, P O BOX 5, RALEIGH, NC, 27602, US

(21) Appl. No.: 09/769,925

(22) Filed: Jan. 25, 2001

(51) Int. Cl. A61F 11/06; G10K 11/16; H03B 29/00; H04B 1/00

(52) U.S. Cl. 381/71.4; 381/71.1; 381/71.2; 381/86

(57) ABSTRACT

Different parameters are supplied to a noise reduction algorithm based on the detection of different vehicle conditions, thereby allowing the noise reduction algorithm to process audio input differently depending on the corresponding vehicle conditions, such as speed, motor rpm, radio on/off status, window open/closed status, occupancy and position of vehicle seat(s), or any combination thereof. One or more vehicle conditions are detected by the vehicle and communicated to a noise reduction control device that determines the appropriate noise reduction algorithm parameters to use based on the detected vehicle conditions, advantageously with reference to a plurality of stored sets of noise reduction algorithm parameters previously established through a calibration process and stored in memory. Received audio input from a microphone array associated with the vehicle may be processed by the noise reduction algorithm as adapted by the appropriate parameters and used by a mobile terminal or the vehicle as appropriate.

### BACKGROUND OF THE INVENTION

The present invention relates to audio noise reduction, and more particularly to an audio noise reduction approach that varies a noise reduction algorithm based on detected vehicle conditions.

It is common for wireless communications mobile terminals, such as cellular phones, to operate in acoustically noisy environments. It is further common for wireless communications mobile terminals to have, or operate in conjunction with, noise reduction algorithms designed to reduce the amount of background audio noise included in the signals transmitted by the devices. Typically, these noise reduction algorithms monitor background noise and process the signals based on the audio input at microphone(s) to reduce or eliminate the effect of the background noise.

One common environment where background audio noise is particularly troublesome is in vehicles. It is common for wireless communications mobile terminals in such environments to be mated with a hands-free adapter mounted in the vehicle. In such an arrangement, there is typically a substantial distance between the users mouth and the “normal” microphone of the mobile terminal. This distance allows background noise to be more easily commingled with the user's voice at the “normal” microphone and reduces the apparent intelligibility of the user's voice. To combat this, it is common to disable the input from the “normal” microphone and substitute input from a vehicle mounted microphone instead. The vehicle mounted microphone may be positioned closer to the user's mouth and/or along an acoustically cleaner path from the user's mouth. However, while the use of vehicle mounted microphones may help to improve performance, the overall performance may still be substantially less than ideal. In particular, such approaches tend to focus exclusively on direct measurement of background noise and do not readily adapt to changing physical mechanical/electrical vehicle characteristics, such as a change in the user's seat position.

### BRIEF SUMMARY OF THE INVENTION

The present method and apparatus supplies different noise reduction parameters to a noise reduction algorithm based on the detection of different vehicle conditions, thereby allowing the noise reduction algorithm to process audio input differently depending on the corresponding vehicle conditions. One or more vehicle conditions are detected by the vehicle and communicated to a noise reduction control device. The noise reduction control device determines the appropriate noise reduction algorithm parameters to use based on the detected vehicle conditions, advantageously with reference to a plurality of stored sets of noise reduction algorithm parameters previously established through a calibration process and stored in memory. Examples of vehicle conditions that may impact the noise reduction algorithm include vehicle speed, vehicle motor rpm, vehicle radio on/off status, open/closed status of one or more vehicle windows, occupancy and position of one or more vehicle seats, or any combination thereof, but specifically does not include ambient noise level or the like. Received audio input from a microphone array associated with the vehicle may be processed by the noise reduction algorithm (as adapted by the appropriate parameters). The “noise reduced” audio may then be used to generate an RF signal and/or for voice recognition purposes by either a wireless communications mobile terminal or the vehicle itself.

### BRIEF DESCRIPTION OF THE DRAWINGS

FIG. 1 depicts a vehicle with one seat occupied in the forward position.

FIG. 2 depicts the vehicle of FIG. 1 with the seat in a different position.

FIG. 3 shows one exemplary process flow for the present invention.

FIG. 4 shows one exemplary process flow for establishing calibrated noise reduction algorithm parameters according to the present invention.

### DETAILED DESCRIPTION OF THE INVENTION

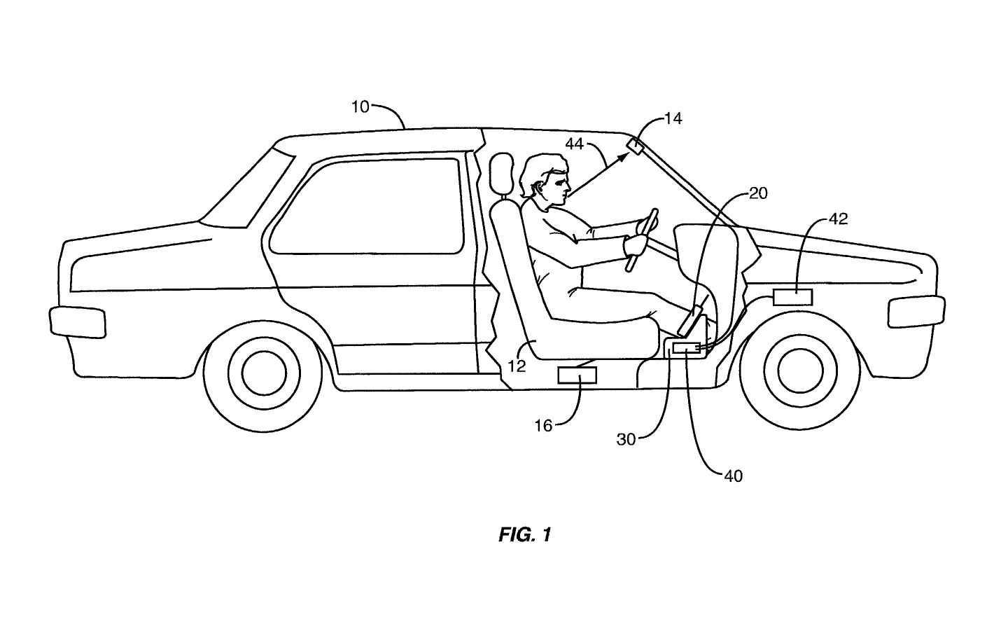

Referring to FIG. 1, a person is shown sitting in a seat 12 of a vehicle 10. While the vehicle 10 shown is a car for simplicity, the vehicle 10 may be any type of vehicle 10, such as a truck, a bus, a boat, a plane, etc. The vehicle 10 includes a microphone array 14 of one or more microphones and at least one vehicle condition detector 16, and advantageously a plurality of vehicle condition detectors 16, whose function is explained further below. Of course, the vehicle 10 may also include a motor, tires, a CD player, and the like, which are generally not shown in FIG. 1 for simplicity as these items are well known in the art.

A wireless communications mobile terminal 20 is shown coupled to a hands-free adapter 30 mounted in the vehicle 10. The user inputs audio, such as speech, to the mobile terminal 20 via the microphone array 14. The inputs to the microphone array 14 are directed to a noise reduction control device 40. The noise reduction control device 40 may be comprised of suitable circuits within the hands-free adapter 30 (as shown) or may be elsewhere in the vehicle 10, such as near the microphone array 14. Regardless, the noise reduction control device 40 utilizes a noise reduction algorithm to process the input from the microphone array 14 and forwards the resulting signal to the mobile terminal 20. The noise reduction algorithm may take any suitable form known in the art. For purposes of illustration, the noise reduction algorithm may be that disclosed in U.S. patent application Ser. No. __/____, filed Jan. 10, 2001, entitled “Noise Reduction Apparatus and Method,” and incorporated herein by reference. The mobile terminal 20 takes the noise-reduced input from the noise reduction control device 40, processes it as appropriate, and transmits an RF signal and/or performs voice recognition functions based thereon. The details of the configuration and operation of the mobile terminal 20, including voice recognition and RF transmission, are well known in the art and unimportant to understanding the present invention; therefore, such details are not discussed further herein.

A noise reduction algorithm may be tailored to a particular situation by supplying appropriate parameters to the noise reduction algorithm. These parameters may be alternatively known as training parameters, calibration parameters, or simply noise reduction algorithm parameters. For ease of reference, the latter term is used herein. Just by way of example, the Green function (G(ω,rᵢ,r₀)) is a noise reduction algorithm parameter of interest for the above-referenced noise reduction algorithm. Depending on the values of these noise reduction algorithm parameters, the noise reduction algorithm will process the incoming audio differently.

The present invention determines the values for the noise reduction algorithm parameters based at least in part on one or more detected vehicle conditions. The term “vehicle conditions” is intended to encompass conditions related to the physical mechanical/electrical condition of the vehicle 10, such as current speed, seat position, and the like. These vehicle conditions may indicate spatial relationships that may exist within the vehicle 10 relevant to noise reduction, such as seat positions or window open/close status (potential sound reflective surface available or not), or otherwise indirectly help predict a noise field within the vehicle 10, such as radio on/off status, but are not direct measurements of the ambient noise in the vehicle 10 (as this is not a physical mechanical/electrical condition of the vehicle 10). As such, the term “vehicle conditions,” as used herein, is intended to exclude characteristics not related to the physical mechanical/electrical condition of the vehicle—such as direct measurements of ambient noise level or the like—and is instead intended to include characteristics related to the physical mechanical/electrical condition of the vehicle—such as vehicle speed, vehicle motor rpm, vehicle radio on/off status, open/closed status of one or more vehicle windows, position of one or more vehicle seats 12, occupancy of one or more vehicle seats 12, and the like, or any combination thereof.

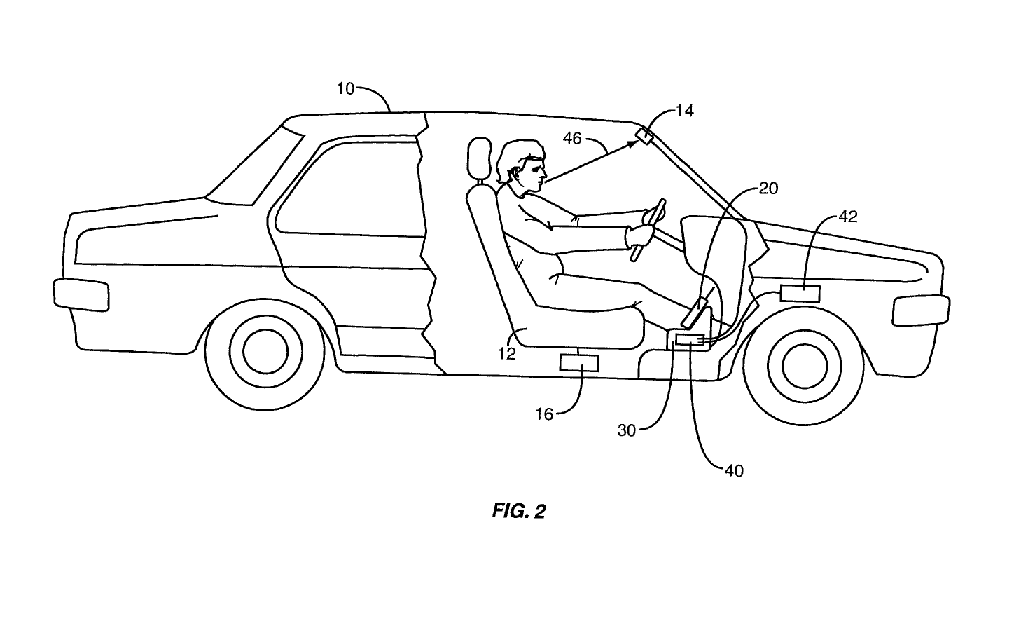

The usefulness of adapting the noise reduction algorithm to the vehicle conditions may be illustrated by comparing FIG. 1 and FIG. 2. In FIG. 1, the seat 12 is show being in a forward position with the seat back substantially vertical. In this arrangement, the path from the user's mouth to the microphone array 14 is indicated at 44. As can be seen, this path is relatively short. Contrast this situation with that shown in FIG. 2, where the seat 12 is shown in a back position with the seat back tilted somewhat back. The path from user's mouth to the microphone in this arrangement is indicated at 46. As can be seen by comparing FIG. 1 and FIG. 2, path 46 is longer than path 44. Accordingly, the amount of noise picked up by microphone array 14 will be larger in FIG. 2, all other things being equal. As such, the noise reduction algorithm may need to process the input from the microphone array 14 in a different fashion for the situation depicted in FIG. 1 than for the situation depicted in FIG. 2. Likewise, the noise reduction algorithm may need to process the audio signal differently depending on whether a window is up or down, the CD player is on or off, the motor is running high rpms or low rpms, and the like, including combinations of various vehicle conditions. The desired variation in the noise reduction algorithm is achieved by determining the values of the noise reduction algorithm parameters based on the vehicle conditions in the present invention.

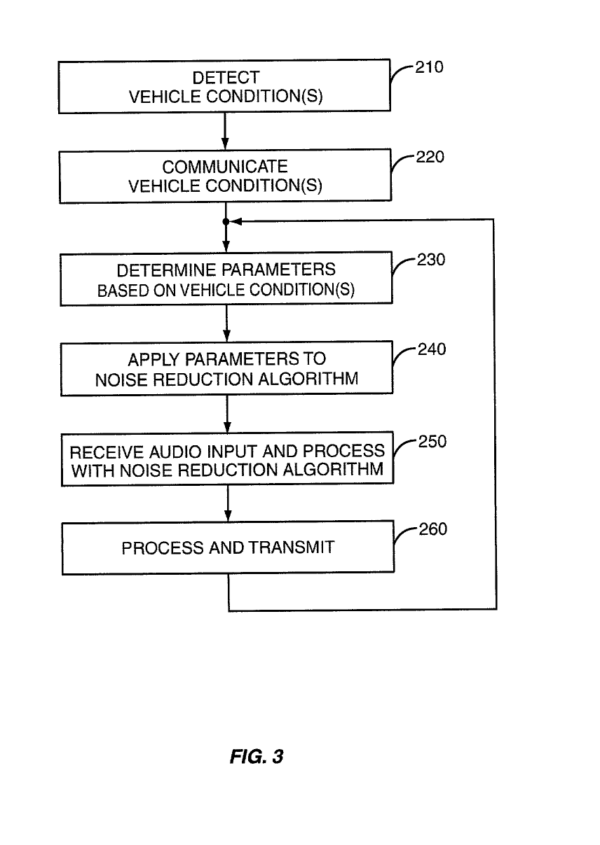

The overall process of the present invention is shown in FIG. 3. The process begins with the vehicle 10 detecting one or more vehicle conditions via the vehicle condition detectors 16 (block 210). For instance, the vehicle 10 may monitor the position of a six-way adjustable driver's seat 12 via suitable detectors 16. The detectors 16 may be mechanical strain gages, electrical capacitive, optical interrupt, or any other know detector apparatus, with the details being a matter of design choice. The vehicle condition detectors 16 may advantageously be already present in the vehicle 10 for other purposes or may be added thereto for this purpose. The status or readings of the detector(s) 16 are communicated to the noise reduction control device 40 (block 220), either directly or through an intervening device, such as the vehicle's overall controller (not shown). The noise reduction control device 40 determines one or more noise reduction algorithm parameters based on the reported vehicle conditions (block 230). For instance, the noise reduction control device 40 may compare the reported vehicle conditions against a look-up table that indicates the set of noise reduction algorithm parameters to be used for each combination of vehicle conditions. One process for providing such stored sets of noise reduction algorithm parameter values is discussed further below. The noise reduction control device 40 then adapts the noise reduction algorithm to the vehicle conditions by applying the identified noise reduction algorithm parameter values to the noise reduction algorithm (block 240). The audio input received at the microphone array 14 is thereafter processed by the noise reduction algorithm as adapted by the supplied noise reduction algorithm parameters (block 250) to produce a noise-reduced audio signal. It should be understood that the noise-reduced audio signal is typically electronic in nature (i.e., not acoustic); however, this signal is referred to as the noise-reduced audio signal because it contains audio data and has been subjected to a noise reduction process. The noise-reduced audio signal is then forwarded from the noise reduction control device 40 to the mobile terminal 20, where it is processed in a conventional fashion to form an RF signal that is transmitted (block 260). Of course, as mentioned above, the noise-reduced audio signal may also or alternatively be used for voice recognition purposes known in the art. The process of adapting the noise reduction algorithm may terminate at that point or may advantageously loop back to adapt to any changes in the detected vehicle conditions (block 230). The detection of vehicle conditions (block 210) and/or the communication of those conditions to the noise reduction control device 40 (block 220) may advantageously occur on a continuous or periodic basis.

Because a given noise reduction algorithm may be used in a variety of vehicles 10, it may be advantageous to calibrate the noise reduction algorithm parameters to a particular vehicle 10, or model of vehicle 10, and store the calibrated noise reduction algorithm parameters in memory 42 associated with the vehicle 10. For instance, a car manufacturer may offer the microphone array 14 and hands-free adapter 30 as an available option for new car purchasers. The car manufacturer can run tests, such as those outlined below, and pre-program appropriate noise reduction algorithm parameter value sets into the non-volatile memory 42 referenced by the respective noise reduction control device 40 installed in the vehicle 10 during manufacture. While the car manufacturer may run tests for each individual vehicle 10, it may be advantageous to run tests on a representative sample of a given vehicle model (e.g., BMW 328is) and the value sets derived therefrom used for all vehicles 10 of that model. The vehicle manufacturer may then supply the vehicle to the vehicle dealer with the vehicle pre-programmed for the noise reduction method of the present invention, or the vehicle dealer may add the memory 42 having the stored sets of noise reduction parameters as a dealer-upgrade. Regardless of the approach, the vehicle 10 can be trained to vary the noise reduction algorithm based on various vehicle conditions without the user having to engage in a complicated training process to “learn” the vehicle's characteristics under various vehicle conditions.

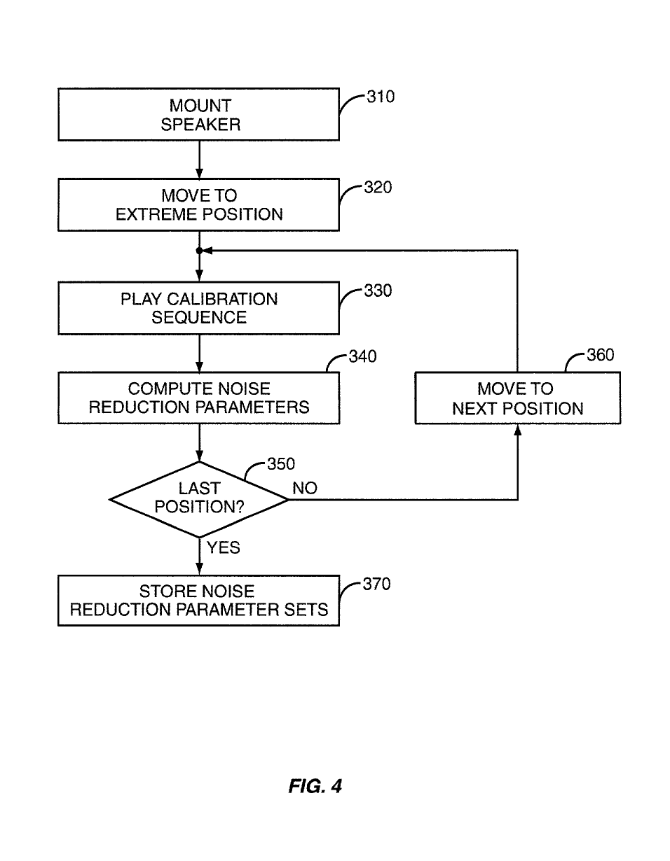

One process for establishing the proper noise reduction algorithm parameter value sets to “train” or “calibrate” the noise reduction algorithm to the possible combinations of vehicle conditions for a particular vehicle 10 is shown is FIG. 4. For simplicity, the present example will use seat position of a single seat 12 as the relevant vehicle condition, but the approach can easily be used for other vehicle conditions or combinations of vehicle conditions. A loudspeaker is mounted in the driver's seat 12 at an average person's mouth height, optionally using a suitably sized dummy (block 310). The seat 12 is moved to a first extreme position, for example all the way to the front (block 320). A calibration sequence is played through the loudspeaker and detected by the microphone array 14 (block 330). The data from the microphone array 14 is forwarded to a suitable computer to compute the appropriate noise reduction algorithm parameters for this situation (block 340). These values are cross-referenced to the corresponding vehicle conditions (e.g., driver's seat at position X) as reported by the vehicle condition detectors 16. If there are additional seat positions to be tested (block 350), the seat 12 is then moved to another position (block 360) and steps 330-360 are repeated until all possible seat positions are visited. The collection of cross-referenced noise reduction algorithm parameter sets are then stored for later use (block 370). Suitable non-volatile memory 42, such as a ROM, flash memory, or the like, is then programmed with the noise reduction algorithm parameter sets and associated with a vehicle 10. Thereafter, when the vehicle condition detectors 16 report a certain set of vehicle conditions to the noise reduction control device 40, the noise reduction control device 40 may look-up the appropriate noise reduction algorithm parameters by referencing the memory 42, finding the noise reduction algorithm parameter set that corresponds to the reported vehicle conditions, and change the noise reduction algorithm accordingly. The noise reduction algorithm parameter sets may of course also be used for other vehicles of the same model.

Continuing with the seat position example, the process outlined above works well for seats 12 that are limited to a few discrete possible positions. However, some vehicles 10 allow for continuous adjustment of the seat 12 front-to-back and/or up/down, etc. within certain limits. For such vehicles 10, testing to establish corresponding noise reduction algorithm parameters for all the available seat locations, and/or allocating sufficient memory 42 space to store a very large number of sets of noise reduction algorithm parameters, may be problematic. Thus, it may be advantageous to test only a finite set of the available seat positions and use the data for the finite set to determine the appropriate noise reduction algorithm parameters for the entire spectrum of seat positions. This determination may be through a “nearest neighbor” approach where the noise reduction algorithm parameter set for the tested seat position spatially nearest the reported seat position (vehicle condition) is chosen. Alternatively, a weighted combination of sets may be employed such that the noise reduction algorithm parameter sets for the two closest tested positions are combined in weighted fashion according to the proximity to the reported position.

The discussion above has assumed that the noise reduction control device 40 is incorporated into the hands-free adapter 30; however, this is not required. Indeed, the noise reduction control device 40 may be associated with the vehicle 10 in any manner and may be separate from the hands-free adapter 30 if desired. Further, a hands-free adapter 30 in the conventional sense is not per se required. For instance, the mobile terminal 20 may plug into a port on the vehicle 10, and thereby access the microphone array 14 and the noise reduction control device 40. Likewise, the microphone array 14 and memory 42 associated with the vehicle 10 may be a portion of the vehicle 10 directly or may be a portion of the hands-free adapter 30 that is mated to the vehicle 10.

As used herein, the term “mobile terminal” 20 may include a cellular radiotelephone with or without a multi-line display; a Personal Communications System (PCS) terminal that may combine a cellular radiotelephone with data processing, facsimile and data communications capabilities; a Personal Digital Assistant (PDA) that can include a radiotelephone, pager, Internet/intranet access, Web browser, organizer, calendar and/or a global positioning system (GPS) receiver; and a conventional laptop and/or palmtop receiver or other appliance that includes a radiotelephone transceiver. Mobile terminals 20 may also be referred to as “pervasive computing” devices.

The discussion above has generally assumed the presence of a wireless communications mobile terminal 20; however, the present invention also encompasses situations where a mobile terminal 20 is not present. For instance, the noise reduction control device 40 may be associated with the vehicle 10 and the vehicle 10 itself may use the noise reduced audio signal for voice recognition purposes, such as to recognize commands (e.g., “tune radio to station X”), user identity, driver sobriety, and the like. The uses and details of voice recognition are well known in the art and further detailed discussion thereof is omitted. By supplying a “cleaner” audio signal to the voice recognition hardware/software, the approach of the present invention may help improve the overall functioning of the voice recognition operation.

The present invention may, of course, be carried out in other specific ways than those herein set forth without departing from the scope of the invention. The present embodiments are, therefore, to be considered in all respects as illustrative and not restrictive, and all changes coming within the meaning and equivalency range of the appended claims are intended to be embraced therein.

What is claimed is:

1. A method of reducing audio noise transmitted by a wireless communications mobile terminal associated with a vehicle, comprising:
detecting one or more vehicle conditions by said vehicle, wherein said vehicle conditions relate to the position of one or more seats within said vehicle;
communicating said vehicle conditions to a noise reduction control device associated with said vehicle;
determining one or more noise reduction algorithm parameters based on said vehicle conditions by said noise reduction control device by referencing a plurality of sets of noise reduction parameters previously stored in memory associated with said vehicle, said sets corresponding to a plurality of vehicle conditions;
adapting a noise reduction algorithm in response to said vehicle conditions by applying said noise reduction algorithm parameters to vary said noise reduction algorithm;
receiving audio input at a microphone array associated with said vehicle; and
processing said audio according to said noise reduction algorithm as adapted by said noise reduction algorithm parameters and transmitting an RF signal based thereon.

2. The method of claim 1 wherein determining one or more of said noise reduction parameters based on said detected vehicle conditions comprises selecting one of said stored sets of noise reduction parameters and wherein applying said noise reduction parameters to vary said noise reduction algorithm comprises applying said selected set of noise reduction parameters to vary said noise reduction algorithm.

3. The method of claim 2 wherein selecting one of said stored sets of noise reduction parameters comprises selecting the stored set of noise reduction parameters that corresponds closest to said detected vehicle conditions.

4. The method of claim 1 wherein determining said noise reduction parameters based on said detected status comprises selecting more than one of said stored sets of noise reduction parameters based on said vehicle conditions and performing a weighted combination thereof.

5. The method of claim 1 wherein said vehicle is a first vehicle and wherein said microphone array is a first microphone array associated with said first vehicle and further comprising:
associating a second microphone array with a second vehicle;
gathering noise reduction calibration data for a plurality of vehicle conditions using said second vehicle and said second microphone array;
computing reference noise reduction parameters for a plurality of vehicle conditions based on said calibration data;
storing sets of said reference noise reduction parameters in memory with corresponding vehicle condition indicators; and
associating said memory with said first vehicle.

6. A method of providing calibration parameters to a noise reduction algorithm associated with a second vehicle, comprising:
gathering noise reduction calibration data for a plurality of vehicle conditions using a first vehicle;
computing reference noise reduction parameters for a plurality of vehicle conditions based on said calibration data;
storing sets of said reference noise reduction parameters in memory with corresponding vehicle condition indicators; and
associating said memory with a second vehicle prior to first possession of said second vehicle by any retail customer.

7. The method of claim 6 wherein associating said memory with said second vehicle prior to first possession of said second vehicle by any retail customer comprises associating said memory with said second vehicle during manufacture of said second vehicle.

8. The method of claim 6 wherein said vehicle conditions relate to the position of one or more seats within said vehicle.

9. The method of claim 8 wherein said vehicle conditions relate to the occupancy and position of one or more seats within said vehicle.

10. The method of claim 6 wherein said vehicle conditions relate to one or more of the characteristics selected from the group consisting of vehicle speed, vehicle motor rpm, vehicle radio on/off status, open/closed status of one or more vehicle windows, position of one or more vehicle seats, and any combination thereof.

11. The method of claim 6 wherein gathering said noise reduction calibration data for said plurality of vehicle conditions using said first vehicle comprises gathering said noise reduction calibration data for said plurality of vehicle conditions using a first microphone array associated with said first vehicle.

12. The method of claim 11 wherein said microphone array comprises more than one microphone.

13. A method of generating a noise reduced audio signal, comprising:
detecting one or more vehicle conditions by a vehicle;
communicating said vehicle conditions to a noise reduction control device associated with a said vehicle;
determining one or more noise reduction algorithm parameters based on said vehicle conditions by said noise reduction control device;
adapting a noise reduction algorithm in response to said vehicle conditions by applying said noise reduction algorithm parameters to vary said noise reduction algorithm;
receiving audio input at a microphone array associated with said vehicle; and
processing said audio according to said noise reduction algorithm as adapted by said noise reduction algorithm parameters to produce said noise reduced audio signal.

14. The method of claim 13 further comprising storing a plurality of sets of noise reduction parameters in memory associated with said vehicle prior to said detecting, said sets corresponding to a plurality of vehicle conditions; and wherein determining one or more of said noise reduction parameters based on said detected vehicle conditions comprises referencing said stored sets.

15. The method of claim 14 wherein determining one or more of said noise reduction parameters based on said detected vehicle conditions comprises selecting one of said stored sets of noise reduction parameters and wherein applying said noise reduction parameters to vary said noise reduction algorithm comprises applying said selected set of noise reduction parameters to vary said noise reduction algorithm.

16. The method of claim 15 wherein selecting one of said stored sets of noise reduction parameters comprises selecting the stored set of noise reduction parameters that corresponds closest to said detected vehicle conditions.

17. The method of claim 14 wherein determining said noise reduction parameters based on said detected status comprises selecting more than one of said stored sets of noise reduction parameters based on said vehicle conditions and performing a weighted combination thereof.

18. The method of claim 13 wherein said vehicle conditions relate to the position of one or more seats within said vehicle.

19. The method of claim 18 wherein said vehicle conditions relate to the occupancy and position of one or more seats within said vehicle.

20. The method of claim 13 wherein said vehicle conditions relate to one or more of the characteristics selected from the group consisting of vehicle speed, vehicle motor rpm, vehicle radio on/off status, open/closed status of one or more vehicle windows, position of one or more vehicle seats, and any combination thereof.

21. The method of claim 13 further comprising transmitting an RF signal based on said noise reduced audio signal.

22. The method of claim 13 wherein said vehicle is a first vehicle and wherein said microphone array is a first microphone array associated with said first vehicle and further comprising:
associating a second microphone array with a second vehicle;
gathering noise reduction calibration data for a plurality of vehicle conditions using said second vehicle and said second microphone array;
computing reference noise reduction parameters for a plurality of vehicle conditions based on said calibration data;
storing sets of said reference noise reduction parameters in memory with corresponding vehicle condition indicators; and
associating said memory with said first vehicle
wherein adapting said noise reduction algorithm in response to said vehicle conditions comprises communicating at least one of said sets of said reference noise reduction parameters from said memory to said noise reduction control device associated with said vehicle.

23. A method of generating a noise reduced audio signal, comprising:
providing a noise reduction algorithm modifiable based on predefined noise reduction algorithm parameters;
detecting one or more vehicle conditions by a vehicle;
selecting a set of noise reduction algorithm parameters from a plurality of stored sets of noise reduction algorithm parameters based on said detected conditions; said stored sets being stored in a memory associated with said vehicle;
modifying said noise reduction algorithm in response to said vehicle conditions based on said selected set of noise reduction algorithm parameters;
receiving audio input at a microphone array associated with said vehicle; and
processing said audio according to said noise reduction algorithm as adapted by said noise reduction algorithm parameters to produce said noise reduced audio signal.

24. The method of claim 23 wherein said vehicle conditions relate to the position of one or more seats within said vehicle.

25. A method of generating a noise reduced audio signal, comprising:
processing received audio input by a noise reduction algorithm that varies according to values assigned to a plurality of noise reduction algorithm parameters; and
detecting one or more vehicle conditions by a vehicle;
assigning values to said noise reduction algorithm parameters based on both said detected vehicle conditions and at least one previously stored set of noise reduction algorithm parameters;
receiving audio input at a microphone array associated with said vehicle; and
processing said audio according to said noise reduction algorithm as adapted by said noise reduction algorithm parameters to produce said noise reduced audio signal.

26. The method of claim 25 further comprising storing a plurality of sets of noise reduction parameters in memory associated with said vehicle prior to said detecting, said sets corresponding to a plurality of vehicle conditions; and wherein assigning values to said noise reduction algorithm parameters based on both said detected vehicle conditions and at least one previously stored set of noise reduction algorithm parameters comprises referencing said stored sets.

27. The method of claim 26 wherein assigning values to said noise reduction algorithm parameters based on both said detected vehicle conditions and at least one previously stored set of noise reduction algorithm parameters comprises selecting one of said stored sets of noise reduction parameters.

28. The method of claim 27 wherein selecting one of said stored sets of noise reduction parameters comprises selecting the stored set of noise reduction parameters that corresponds closest to said detected vehicle conditions.

29. The method of claim 26 wherein assigning values to said noise reduction algorithm parameters based on both said detected vehicle conditions and at least one previously stored set of noise reduction algorithm parameters comprises selecting more than one of said stored sets of noise reduction parameters based on said vehicle conditions and performing a weighted combination thereof.

30. A noise reduction control apparatus adapted to determine one or more noise reduction algorithm parameters based on received information indicative of at least one vehicle condition and to process audio input with a noise reduction algorithm that varies according to said noise reduction algorithm parameters.

31. The apparatus of claim 30 further comprising a microphone array associated with a first vehicle and communicating said audio input to said noise reduction control device.

32. The apparatus of claim 31 wherein said microphone array comprises more than one microphone.
